(19) United States
(12) Patent Application Publication
(10) Pub. No.: US 2021/0382048 A1
(43) Pub. Date: Dec. 9, 2021

(54) DEVICES AND METHODS FOR THERAPEUTIC DRUG MONITORING

(71) Applicant: TRITON SYSTEMS, INC., Chelmsford, MA (US)

(72) Inventors: Baris UNAL, Lowell, MA (US); Gulden CAMCI-UNAL, Lowell, MA (US); Darlin LANTIGUA, Lawrence, MA (US)

(21) Appl. No.: 17/339,295

(22) Filed: Jun. 4, 2021

Related U.S. Application Data
(60) Provisional application No. 63/034,722, filed on Jun. 4, 2020.

(51) Int. Cl. G01N 33/543

(52) U.S. Cl. CPC G01N 33/54386; G01N 33/54306

(57) ABSTRACT

Devices and methods to perform a competitive immunoassay are disclosed. In some embodiments, the competitive immunoassay is for the detection of tacrolimus. The devices comprise a plurality of layers optionally made of cellulose-based material, wherein the plurality of layers comprises at least one read-out layer displaying a colorimetric readout of the device to indicate the result of the test to the user.

### CROSS REFERENCE TO RELATED APPLICATIONS

This application claims priority to U.S. Provisional Application Ser. No. 63/034,722 filed on Jun. 4, 2020 the content of which is incorporated herein by reference in its entirety.

### GOVERNMENT INTEREST

This invention was made with Government support from the Department of the Army, Medical Research Acquisition, under Contract No. W81XWH-19-C-0084. The Government has certain rights in this invention.

### BACKGROUND

Tacrolimus is one of the most effective drugs in combating Vascularized Composite Allotransplantation (VCA) rejection. Tacrolimus is an immunosuppressant that inhibits cytokine production and blocks cell division, in addition to inhibiting both interleukin (IL-2) production and the expression of the IL-2 receptors. It is the most common calcineurin inhibitor (CNI), which help shut down T-cell activation in the immune system. These T-cell and IL-2 inhibitions make tacrolimus an invaluable drug for preventing rejection of tissues or organs such as the heart. The applications of tacrolimus extend beyond this, however, because it has recently been shown as highly effective in applications for VCA research (transplanting tissues such as bone, muscle, nerve, and skin to a patient with a substantial injury from a deceased donor of the same species). While immunosuppressant medicine is used to combat the initial attack against the foreign tissue of these grafts, a long-term defense against this attack is required, hence the importance of using maintenance immunosuppression drugs. Tacrolimus, in combination with mycophenolate mofetil (MMF), mycophenolate sodium, azathioprine (AZA), sirolimus, and steroids, provides these desired effects.

While VCA transplants include life enhancing surgeries, there is a severe risk of graft rejection. Tacrolimus reduces this risk because with a sufficiently high concentration (about 5-10 ng/mL), the patient's immune system can be rendered ineffective in its plight to attack the foreign tissue. However, if the tacrolimus concentration is not elevated enough (about <5 ng/mL), the transplanted tissue is attacked and possibly destroyed by the patient's immune system. Tacrolimus has a narrow therapeutic range, and a slightly superfluous amount of it (about >20 ng/mL) can result in numerous side effects, such as renal blood flow and creatinine clearance, microangiopathic hemolytic anemia, hypertension, central-nervous-system demyelination, decrease in pancreatic insulin production, and nephrotoxicity. Common techniques for determining the concentrations of tacrolimus in blood include LC-MS/MS, enzyme-multiplied immunoassay (EMIT), radioimmunoassay (ACMIA), and electrochemiluminescence immunoassay (ELICA). While LC-MS/MS has been identified as the gold standard, it has the potential for cross-reactivity between parent drug and metabolites. This falsely elevated concentration value, in addition to the cost and labor required to complete the procedure, renders it undesirable. LC-MS also requires highly-trained specialists to use and evaluate the results. The EMIT suffers from nonspecific cross-reactivity, leading to poor repeatability between analytical runs, as well as a wide dispersion of results in proficiency testing. Besides these drawbacks, EMIT uses expensive reagents. The ACMIA has several weaknesses too, including insufficient functional sensitivity, inaccuracy at low analyte concentrations, and shift of assay results over time. Electrochemiluminescence immunoassay (ECLIA) has higher cross-reactivity than ACMIA, and there is up to an 11% bias between ELCIA and LC-MS/MS. Another disadvantage of electrochemiluminescence is the need for specialized instrumentation that can induce generation of electrochemically-excited states coupled with sensitive light detection. The limitations of the currently available approaches require a new technique to be formulated—a low-cost and simple platform for detection and quantification of tacrolimus from human blood.

Therefore, there is a need for devices and methods for the rapid detection and quantification of immunosuppressants such as tacrolimus in human blood.

### SUMMARY

The disclosure describes devices and methods to perform a competitive immunoassay assay, such as ELISA, are disclosed. The disclosed embodiments include:

In one embodiment, there is a device for performing a competitive immunoassay assay, the device comprising a plurality of layers each comprising one or more hydrophilic regions, one or more hydrophilic channels, or a combination thereof embedded in the layers. In this embodiments, the one or more hydrophilic channels are fluidically connected to the one or more hydrophilic regions. In this embodiment, the plurality of layers comprises a sample pad layer, a plasma separation membrane layer, a conjugate layer, an incubation layer, a test read-out layer, and a blotting layer. In this embodiment, the conjugate layer comprises at least two hydrophilic regions each comprising colloidal gold. In this embodiment, the test read-out layer comprises at least a first hydrophilic region and a second hydrophilic region, the first hydrophilic region comprises a reagent, and the second hydrophilic region optionally comprises a reagent selected from the group consisting of antigens and antibodies.

In another embodiment, the fluid sample is a serological sample.

In another embodiment, the serological sample is a blood sample.

In another embodiment, the one or more of the layers are cellulose-based layers.

In another embodiment, the competitive immunoassay assay is for the detection of tacrolimus.

In another embodiment, the colloidal gold is conjugated with anti-FK-506 antibodies.

In another embodiment, the reagent from the first hydrophilic region of the read-out layer is BSA-FK 506 conjugate.

In another embodiment, the reagent from the second hydrophilic region of the read-out layer is an antibody, and the antibody is an anti-IgM antibody or anti-IgG antibody

In another embodiment, the two hydrophilic regions of the conjugate layer further comprise a first buffer, the first hydrophilic region of the read-out layer comprises a second buffer, and the second hydrophilic region of the read-out layer comprises a third buffer.

In another embodiment, the first, second, and third buffers comprise DPBS buffer.

### BRIEF DESCRIPTION OF THE DRAWINGS

Aspects, features, benefits and advantages of the embodiments described herein will be apparent with regard to the following description, appended claims, and accompanying drawings where: The present disclosure is described with reference to the following figures, which are presented for the purpose of illustration only and are not intended to be limiting.

In the drawings:

FIG. 1A is a schematic representation of a fabrication of a six-layer paper-based point-of-care device using a wax printing method in accordance with aspects of the present disclosures;

FIG. 1B is a schematic representation of: (i) a Tacrolimus-BSA and control Ab immobilizations on a detection zone and a control zone of a microfluidic device at a test-readout layer; and (ii) a competitive assay results' determination for a positive result and a negative result in accordance with aspects of the present disclosures;

FIG. 1C is a schematic representation of a colorimetric results interpretation on images of test results taken by an android cellphone and a quantification of the test results using ImageJ processing software in accordance with aspects of the present disclosures;

FIG. 2A are a schematic representation of a procedure, images of results, and a diagram of tests quantifications for a colorimetric detection and quantification of tacrolimus in whole human blood at 25, 10 and 1 ng/mL concentrations in accordance with aspects of the present disclosures;

FIG. 2B are images showing tests results and a diagram showing tests quantifications for a colorimetric detection and quantification of tacrolimus in whole human blood at 100, 21, 10, 4 and 0 ng/mL concentrations in accordance with aspects of the present disclosures;

FIG. 3A are images showing colorimetric results for devices containing whole human blood spiked with 10 ng/mL tacrolimus in accordance with aspects of the present disclosures;

FIG. 3B is a diagram showing a quantification of tacrolimus on paper-based microfluidic devices in an aging experiment stored at 50° C. in accordance with aspects of the present disclosures;

FIG. 4A are images showing interference test results for a colorimetric detection of tacrolimus in the presence of Sirolimus (Rapamycin) and Mycophenolate Mofetil in accordance with aspects of the present disclosures;

FIG. 4B is a diagram showing quantification results of tacrolimus tested in the presence of potentially interfering co-administered drugs (sirolimus and mycophenolate mofetil) in accordance with aspects of the present disclosures;

FIG. 4C are images showing interference test results for a colorimetric detection of tacrolimus in the presence of endogenous substances (Bilirubin, Cholesterol, Uric acid, Albumin, and Gamma globulin) in accordance with aspects of the present disclosures; and

FIG. 4D is a diagram showing quantification results of tacrolimus in the presence of potentially interfering endogenous substances in accordance with aspects of the present disclosures.

### DETAILED DESCRIPTION

It will be appreciated that for clarity, the following discussion will describe various aspects of embodiments of the applicant's teachings, while omitting certain specific details wherever convenient or appropriate to do so. For example, discussion of like or analogous features in alternative embodiments may be somewhat abbreviated. Well-known ideas or concepts may also for brevity not be discussed in any great detail. The skilled person in the art will recognize that some embodiments of the applicant's teachings may not require certain of the specifically described details in every implementation, which are set forth herein only to provide a thorough understanding of the embodiments. Similarly, it will be apparent that the described embodiments may be susceptible to alteration or variation according to common general knowledge without departing from the scope of the disclosure. The following detailed description of embodiments is not to be regarded as limiting the scope of the applicant's teachings in any manner.

Various terms are used herein consistent with their common meanings in the art. The following terms are defined below for clarity.

It must also be noted that as used herein and in the appended claims, the singular forms “a”, “an”, and “the” include plural reference unless the context clearly dictates otherwise. Thus, for example, reference to “a device” is a reference to “one or more devices” and equivalents thereof known to those skilled in the art, and so forth.

As used herein, the term “about” means plus or minus 10% of the numerical value of the number with which it is being used. Therefore, “about 50” means in the range of 45-55.

It will be appreciated that while a particular sequence of steps is shown and described herein for purposes of explanation, the sequence may be varied in certain respects, or the steps may be combined, while still obtaining the desired configuration. Additionally, modifications to the disclosed embodiment and the invention as claimed are possible and within the scope of this disclosed invention.

The embodiments described herein are directed specifically to tacrolimus as an example, the methods, and devices described herein are applicable to other immunosuppressants and therapeutic drugs as well. Exemplary immunosuppressants include but are not limited to tacrolimus, cyclosporine, mycophenolate mofetil, mycophenolate sodium, azathioprine, sirolimus, prednisone.

Some embodiments of the invention are directed to therapeutic drug monitoring (TDM) devices and methods for performing tests for tacrolimus levels in serological samples. In some embodiments, the serological sample is a blood sample. As discussed in more detail below, in some embodiments, devices and methods for performing competitive immunoassay point-of-care (POC) tests for tacrolimus levels in human blood samples are disclosed.

The devices disclosed herein comprise a plurality of layers each comprising one or more hydrophilic regions, one or more hydrophilic channels, or a combination thereof embedded in the layers. The hydrophilic channels are fluidically connected to the hydrophilic regions. The plurality of layers comprises at least a sample pad layer, a plasma separation membrane layer, a conjugate layer, an incubation layer, a test read-out layer, and a blotting layer. The conjugate layer comprises at least two hydrophilic regions each comprising colloidal gold. The test read-out layer comprises at least a first hydrophilic region and a second hydrophilic region, and the first hydrophilic region comprises a reagent and the second hydrophilic region optionally comprises a reagent selected from the group consisting of antigens and antibodies. The fluid sample then wicks through the one or more hydrophilic regions and one or more hydrophilic channels to reach the test read-out layer where the competitive immunoassay takes place.

One skilled in the art will appreciate that the analytical capabilities necessary for reliable tacrolimus TDM devices and methods include, without limitation: (i) the ability to detect tacrolimus in whole human blood at ranges relevant to the TDM (about 5-20 ng/mL) to ensure safe yet effective drug levels in the body, (ii) quantification of results relevant to the TDM range to indicate necessary adjustments, and/or (iii) demonstration of these analytical targets to be achieved with time frames and sample volumes relevant to minimally invasive POC diagnostics (e.g. about <50 μL and about <10 minutes). In some embodiments, the device disclosed herein was evaluated by accelerated shelf life testing to ensure proper shelf life parameters. In some embodiments, the effects of potential interferents on assay performance from the device disclosed herein were characterized to ensure high performance.

### Device Design for Simple Tacrolimus POC Bodily Fluids Diagnosis

Sensitive, low-cost, and portable devices are desirable because, for example, they: (i) are less invasive, (ii) require a small amount of time to take and analyze samples, (iii) provide instant warnings at the indication of potential toxicity, (iv) are able to reduce time used by clinical personnel during diagnosis, and/or (v) are able to improving the clinical performance during therapeutic drug monitoring (TDM). However, the need for pre-treatment of samples before detection of the targeted analyte, and the lack of quantification capabilities by the diagnostic tool are the most common obstacles present by immunoassays implemented for TDM.

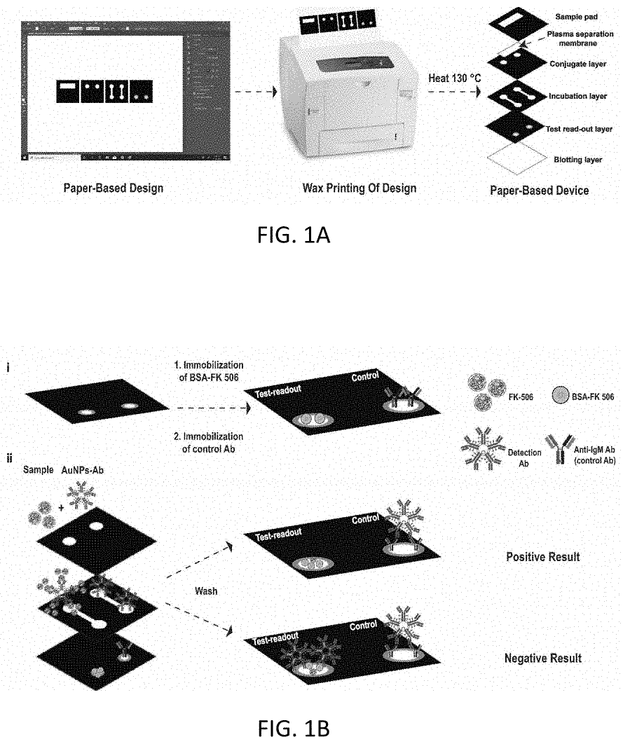

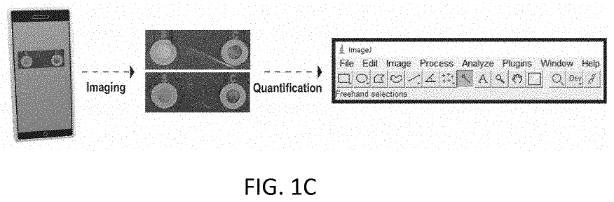

In some embodiments, by implementing the principles of both competitive immunoassays and vertical flow microfluidics, a rapid POC paper-based device is disclosed for colorimetric detection and quantification of tacrolimus in human bodily fluids, such as but not limited to blood, saliva, nasal fluid, mucus, sweat, urine. An example of such device is illustrated in FIG. 1. Specifically, FIG. 1A discloses a schematic representation of the fabrication of a six-layer paper-based POC device using a wax printing method. FIG. 1B discloses Tacrolimus-BSA and control Ab immobilization on the detection and control zones of the microfluidic device at the test-readout layer (section (i), and competitive assay results' determination (positive and negative) (section (ii)). FIG. 1C discloses colorimetric results interpretation on images taken by an android cellphone and quantification of results using ImageJ processing software.

In some embodiments, vertical flow instead of lateral flow is used to wick fluid sample and perform one or more assays within a device. Vertical flow can be achieved using a six-layer device disclosed herein and illustrated in FIG. 1A. One advantage of the vertical flow arrangement is that, for example, the effects of gravity in this arrangement allow for a shorter diagnosis time of the targeted analyte. In addition, it eliminates potential hook effect problems that could compromise the detection efficiency of the diagnostic test. Conversely, a specific anti-tacrolimus antibody with high affinity can be chosen for a competitive application of the immunoassay. Due to the small size of the tacrolimus molecule (MW: 804.031 g/mol) and the presence of few epitopes, using two highly specific antibodies (Abs) for a sandwich immunoassay would prove challenging. Therefore, in some embodiments, a competitive immunoassay approach is preferred.

In some embodiments, there are is interference between test regions in the device disclosed here. Competitive interactions between the targeted analyte (FK-506) and the AuNP-FK506 Mab against the BSA-FK-506 conjugate at the test read-out layer were observed. This was achieved because, at least in part, the red blood cells effectively separated from the unmodified human blood sample in the second layer of the device. In addition, the six-layer vertical flow arrangement of the device disclosed herein allowed for separated interactions between analytes and antibodies at each individual layer (conjugate layer, incubation layer and test read-out layer). Therefore, there are little or no noticeable signal intensity problems at the final test read-out line, which can be observed in lateral flow devices as a result of placement and interference between test lines.

In some embodiments, the cost and colorimetric detection efficiency of the device disclosed herein are prioritized. Specifically, gold nanoparticles of about 20 nm in size were selected to reduce protein concentration during conjugation of the anti-FK506 Ab and increase color intensity during detection. A decrease in the size of gold nanoparticles to about 20 nm is known in the art to require less antibody during conjugation, therefore reducing the cost of the test and improving its scalability as more conjugation solution is obtained by using less antibody. Gold nanoparticles of around 20-40 nm in size are commonly used in the art to develop immunoassays due to their high color intensity and the sensitivity during detection of this particle size.

In some embodiments, the design pattern of the diagnostic device, which can include both a sample pad layer and a blotting layer as part of a six-layer device, allowed for the efficient and rapid detection of tacrolimus under about 10 minutes in non-pretreated human blood. In addition, only about 10 to about 50 μL, for example 20 μL, of sample was required to perform the test. These qualities demonstrate the ability of the device disclosed herein to provide a rapid diagnosis of tacrolimus at a low cost within time frames and sample volumes relevant to minimally invasive POC diagnostics.

### Tacrolimus Therapeutic Drug Monitoring Detection Performance

In some embodiments, the device disclosed herein can detect tacrolimus at concentrations within the drug's standard therapeutic range of about 5-20 ng/mL in unmodified whole human blood sample. Due to the high variability in the blood concentration of tacrolimus among patients, close monitoring of the drug is critical for its effective use in patients that are high-risk, namely, those who are at risk for liver or heart allograft rejection. For these high-risk patients, tacrolimus levels below the recommended therapeutic range may be recommended because low dosages of tacrolimus used with supplemental drugs can reduce the risk of side effects. Because it is important to monitor the concentration of tacrolimus at levels lower than about 3 ng/mL in these high-risk patients, the device disclosed herein can be optimized to detect tacrolimus at concentrations in the higher and lower ends of the recommended drug medical decision range. As a result, concentrations such as about 25 ng/L, about 10 ng/L, and about 1 ng/L were detected and optimized in the device disclosed herein. In addition, concentrations in the ranges of about 100 ng/mL, about 21 ng/mL, about 4 ng/mL, and about 0 ng/mL were also assayed in the device disclosed herein. It is recommended by the International Association of Therapeutic Drug Monitoring and Clinical Toxicology (IATDMCT) that diagnostic devices performing detection of tacrolimus in human blood to show a limit of quantification of about 1 ng/mL to be considered for effective tacrolimus monitoring.

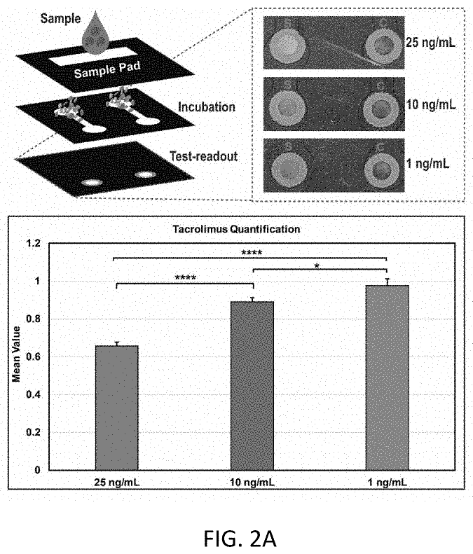

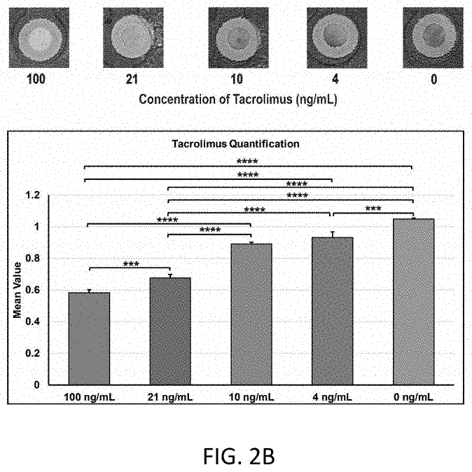

The colorimetric detection and quantification of tacrolimus in unmodified human blood is shown in FIG. 2. Specifically, FIG. 2A shows the colorimetric detection and quantification of tacrolimus in whole human blood at about 25 ng/L, about 10 ng/L, and about 1 ng/L concentrations. The test zone and the control zone were labeled S and C, respectively. Tacrolimus was detected using about 20 μL of whole human blood sample in the test zone S. The detection time was about <10 minutes. When the concentrations of about 25 ng/mL, about 10 ng/mL, and about 1 ng/mL of tacrolimus were tested, the red color formation increased with decreasing tacrolimus concentration, as expected for the competitive assay format. FIG. 2B shows the colorimetric detection and quantification of tacrolimus in whole human blood at about 100 ng/mL, about 21 ng/mL, about 10 ng/mL, about 4 ng/mL and about 0 ng/mL concentrations. Quantification of red color formation for all concentrations was performed using ImageJ. This was done by measuring the grey intensity of images taken using an Android cellphone camera. Three replicates for each condition were performed for all concentration (about 25 ng/mL, about 10 ng/mL, and about 25 ng/mL, A) and (about 100 ng/mL, about 21 ng/mL, about 10 ng/mL, about 4 ng/mL, and about 0 ng/mL, B). Statistical analyses were performed using GraphPad Prism (La Jolla, Calif., USA) (error bars: ±SD, *p<0.05 and ****p<0.0001).

In some embodiments, ImageJ software for quantifying the amount of color formation by images taken on an Android phone were used, and statistical analysis to determine if the results for each concentration were significantly different from each other were performed. As illustrated in FIG. 2A, the formation of color in the test line (S) for the about 25 ng/mL condition is lighter than the about 10 ng/mL and about 1 ng/mL detected concentrations. This result is expected because, at least in part, of the smaller number of free-flowing gold-conjugated antibodies that can travel to the test line when the concentration of tacrolimus in the tested sample is present at a higher concentration. The difference in color intensity is visible to the naked eye in the images for each of these concentrations. In addition, the immense increase in red color formation shown for the smaller concentration that was detected (about 1 ng/mL) demonstrates the colorimetric detection efficiency of the device as a result of the competitive format of the assay. The quantification results for these concentrations shown in FIG. 2A also confirm the statistically significant differences in the color intensity between each concentration (P=<0.0001 and 0.0123, respectively). Additionally, the colorimetric results for the about 100 ng/mL, about 21 ng/mL, about 10 ng/mL, about 4 ng/mL and about 0 ng/mL concentrations in FIG. 2B can also be differentiated by the naked eye. When comparing the colorimetric results for both FIGS. 2A and 2B, the difference in red color intensity across each concentration can be clearly observed, where the about 100 ng/mL concentration shows the lightest color and the concentration with no analyte shows the darkest. The statistical analysis for the quantification results of these concentrations (FIG. 2B) also show significant differences, except between the about 10 ng/mL and about 4 ng/mL concentrations. Significant statistical difference in the quantified color intensity between the no analyte group and all the tested concentrations were observed. Conversely, preliminary pre-calculation results to estimate the within-run precision of the test at each of the optimized detected concentrations (about 25 ng/mL, about 10 ng/mL, and about 1 ng/mL, n=3) were also evaluated and resulted in coefficients of variation (CV) of about 2.8%, about 2.4%, and about 3.5% for each concentration, respectively. This is below the about 20% CV accepted range recommended by the FDA, which indicates an initial value for the repeatability performance of the device.

The embodiments disclosed herein demonstrate the ability of the device disclosed herein to efficiently detect tacrolimus in a small volume of sample (about 20 μL) with differences in the signal intensity and detected concentrations that can be evaluated by the naked eye. One skilled in the art would appreciate that no false positive results were obtained.

### Shelf Life Test

The bioactivity preservation of biomolecules on paper-based devices is a critical factor in determining the effectiveness of a diagnostic device. Parameters such as temperature, humidity, and time are the most critical challenges affecting the shelf-life of a diagnostics device intended to be used in resource limited areas where appropriate storing conditions are extremely lacking.

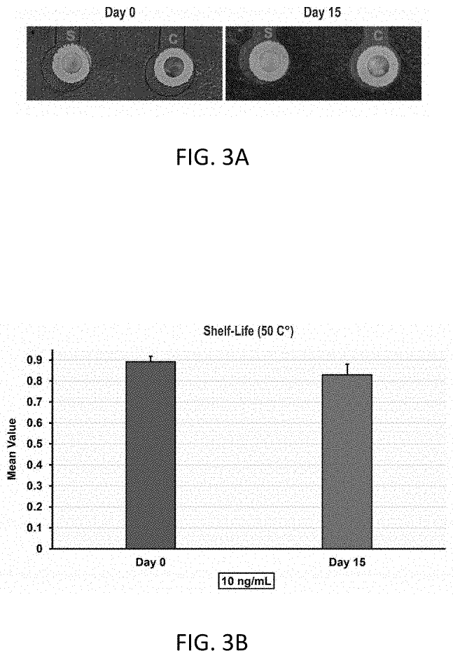

In some embodiments, the shelf-life stability of the device disclosed herein was assessed through accelerated aging testing. FIG. 3 shows the shelf life of tacrolimus detection on the device disclosed herein at about 50° C. Specifically, FIG. 3A discloses colorimetric results for devices containing whole human blood spiked with about 10 ng/mL tacrolimus. The devices were stored at about 50° C. and tested at day 0 and at day 15 of being in storage. Three devices were tested per time point. FIG. 3B shows the quantification of tacrolimus on the device disclosed herein stored at about 50° C.

To provide an initial indication of the real-time shelf-life of the device disclosed herein, devices were stored at about 50° C. for two weeks. The results demonstrate the detection efficacy of the devices was maintained under this rigorous temperature condition when the target analyte (tacrolimus) was detected at about 10 ng/mL concentration as shown in FIG. 3A. In addition, no significant difference was found from the statistical analysis of the quantified data between devices tested at day 0 (0.8909±0.02639) vs. 15 days (0.8287±0.05159) after storage as shown in FIG. 3B. The long-term activity of the dried gold-conjugate anti-tacrolimus antibody on the POC device was preserved in high temperatures (about 50° C.) during the 15-day storing cycle. The stability of the protein was maintained as a result of using sucrose and a blocking agent (casein) to treat the conjugate layer prior to drying the anti-tacrolimus protein on the device's conjugate layer. The supplementation of this choice of reagents prevented the unfolding of the protein during heating. It is well established that the use of non-reducing sugars such as sucrose or trehalose enhance the stability of proteins in high temperatures. In addition, using a mixture of non-reducing sugars and blocking reagents has been shown in the art to preserve the activity of antibodies at temperatures such as about 45° C. in paper-based diagnostic devices used at point-of-care.

The disclosures herein not only validate the detection activity of the device disclosed herein to be preserved at about 50° C. and acceptable under the World Health Organization (WHO) guidelines, but also indicate the real-time shelf-life of the device to be equal to at least about six months at room temperature.

### Assay Performance in the Presence of Potential Interferents

Validation of immunoassay performance under the presence of endogenous compounds and interference drugs must be done to determine the effects of cross reactivity on the efficacy of a diagnostic test. According to the National Committee for Clinical Laboratory Standards “Interference Testing in Clinical Chemistry; Proposed Guideline,” endogenous compounds and commonly co-administered drugs at their highest concentration or 10-fold higher than the highest stablished therapeutic dosage should be tested in the presence of tacrolimus to provide a more accurate understanding for the sensitivity of the diagnostic platform intended for tacrolimus detection.

In some embodiments, to test the influence of interferent drugs and endogenous substances on the detection of tacrolimus, the potential cross reactivity for detecting tacrolimus in the presence of routinely administered medications and physiological compounds is assessed. The interferents compounds used were identified based on recommendations by the Food and Drug Administration (FDA) and National Committee for Clinical Laboratory Standards (NCCLS).

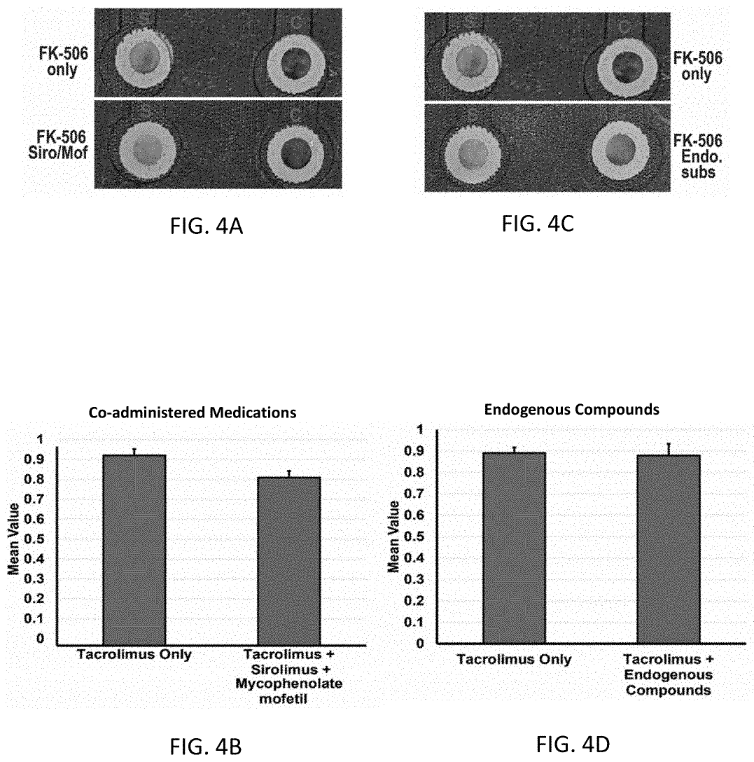

FIG. 4 shows interference tests for Tacrolimus' detection in the presence of co-administered medications and endogenous compounds. Specifically, FIG. 4A shows interference test for the colorimetric detection of tacrolimus in the presence of Sirolimus (Rapamycin), and Mycophenolate Mofetil. Three replicates were carried out for each condition. Colorimetric results for tacrolimus tested alone at about 10 ng/mL concentration in whole human blood are shown in the image labeled as “FK-506 only.” The image labeled as “FK-506, Siro/Mof” demonstrates the results obtained when a mixture containing about 10 ng/mL tacrolimus, about 300 ng/mL sirolimus, and about 100,000 ng/mL mycophenolate mofetil in whole human blood was tested in the devices. FIG. 4B shows quantification results of tacrolimus tested in the presence of potentially interfering co-administered drugs (sirolimus and mycophenolate mofetil). Quantification of red color was performed using ImageJ for the images that were acquired on an Android cellphone camera. The test mixture contained about 10 ng/mL of tacrolimus, about 300 ng/mL sirolimus, and about 100,000 ng/mL mycophenolate mofetil. The statistical analysis results showed no significant difference between devices tested with tacrolimus only and devices tested with tacrolimus in the presence of other interfering drugs (0.8909±0.01524 vs. 0.8438±0.01922). FIGS. 4C and 4D shows the results for the detection of tacrolimus alone (about 10 ng/mL, 0.8909±0.01524) and in the presence of the recommended FDA and NCCLS endogenous substances (about 0.6 mg/mL bilirubin, about 5 mg/mL cholesterol, about 0.2 mg/mL uric acid, about 120 mg/mL albumin, and about 120 mg/mL gamma globulin; 0.8792±0.03131). Image labeled as “FK-506 Endo. Subs” demonstrates the colorimetric results obtained when a mixture containing the tacrolimus, bilirubin, cholesterol, uric acid, albumin, and gamma globulin in whole human blood was tested in the devices. FIG. 4D shows quantification of tacrolimus in the presence of potentially interfering endogenous substances. The statistical analysis results showed no significant difference between devices tested with tacrolimus only and devices tested with tacrolimus in the presence of other interfering endogenous compounds.

As per “Class II Special Controls Guidance Document,” about 10 ng/mL tacrolimus was the preferred concentration tested in the presence of the interferent compounds assayed because, at least in part, it is close to the tacrolimus medical decision level of about 5 ng-15 ng/mL. This concentration was also the known concentration in middle range of test performed, which allows for a lesser chance of bias results and more accurate cross reactivity data. In addition, these results are in accordance with other immunoassay approaches (ECLIA and CMIA), where cross reactivity against interferent compounds such as bilirubin, hematocrit, or total protein is zero. Overall, the results presented herein demonstrate a high effectiveness of the device disclosed herein in the detection of tacrolimus in the presence of interferent substances and co-administered medications.

### EXAMPLES

While several experimental Examples are contemplated, these Examples are intended non-limiting.

### Example 1

### Materials

Whatman chromatography paper, Whatman nitrocellulose membrane, blood separator membrane, blotting paper, the anti-FK-506 Mab, FK-506 (Fujimycin, Tacrolimus) drug, rabbit anti-IgM Ab (control Ab), tacrolimus BSA Conjugate, mycophenolate mofetil, rapamycin from streptomyces, Tween 20, and 3-(N,N-dimethyl myristylammonio) propanesulfonate (Zwittergent), bilirubin, cholesterol soluble in water, human albumin, human gamma globulin, sucrose, uric acid, Dulbecco's phosphate buffer saline (DPBS), blocking solution, double-sided adhesive tape, and whole human blood from healthy donors were used.

### Example 2

### Preparation of Reagents

The blocking buffer and conjugate-layer treatment solutions were prepared using Dulbecco's phosphate-buffered saline (DPBS, 1×) pH about 7.0-7.2 supplemented with Tween 20, sucrose, and casein. The wash buffer was prepared using DPBS (1×) supplemented with Tween 20. The test-line treatment solution was prepared using DI water supplemented with Zwittergent.

### Example 3

### Sample Preparation

The tested samples were prepared using unmodified fresh human blood spiked with tacrolimus (FK-506) alone, tacrolimus in combination with drugs that interfere with tacrolimus detection (sirolimus and mycophenolate mofetil), and lastly, tacrolimus in combination with endogenous substances (bilirubin, cholesterol, uric acid, albumin, and gamma globulin). These prepared samples yielded the desired concentrations tested on the device.

### Example 4

### Selection of Reagent and Synthesis of Colloidal Gold-Anti-Tacrolimus (FK-506) Mab Conjugate (Detector Antibody)

The point-of-care (POC) diagnostic device disclosed herein was fabricated to specifically detect tacrolimus (FK-506), which is a macrolide antibiotic with a reliable immunosuppressive function proven to be effective in combating Vascularized Composite Allotransplantation (VCA) rejection. Conjugation of the Anti-FK-506 Mab to colloidal gold nanoparticles was accomplished by strictly following the DCN Gold Conjugation Kit's protocol. Prior to conjugation, the anti-FK-506 protein was dialyzed.

### Example 5

### Fabrication of POCT Paper-Based Diagnostic Device

A paper-based POC device comprised of six layers arranged in a vertical flow was fabricated using the principles of competitive immunoassays for detecting small molecules. Each of the six layers was designed with Adobe Illustrator. Wax printing was used to establish hydrophobic areas that surround the active hydrophilic regions of the layers and device, and this was done on every layer except the blotting and plasma separation membrane layers. The other four layers of the device were printed using a Xerox ColorQube 8580 wax printer on commercially available Whatman chromatography sheets or nitrocellulose membranes. The sample pad layer, conjugate layer, and incubation layers of the device were printed on Whatman chromatography paper. However, the test readout layer was printed on Whatman nitrocellulose membrane. These paper layers were then baked in an oven at about 130° C. to facilitate melting of the wax (about 30 secs), which created hydrophobic boundaries that defined the sample zones. Finally, the printed paper devices were cut using a guillotine-type paper cutter, and prior to assembling, the conjugate and test read-out layers were treated with reagents. Details of the reagent treatment are disclosed in Example 6. To assemble the layers together, adhesive films patterned with opened holes and channels created from a laser cutter machine were placed on the back-side of each layer. Lastly and as shown in FIG. 1A, the device was constructed by stacking the layers together, starting with the sample pad layer as the first layer and the blotting paper as the final bottom layer. The fully assembled device dimensions are about 1.75 cm by about 1.75 cm.

FIG. 1A shows a schematic representation of a fabrication of a six-layer paper-based POC device using a wax printing method in accordance with aspects of the present disclosure. Specifically, FIG. 1A shows the paper-based design, a wax printing of the design, and an assembly of the paper-based device comprising, without limitation, a sample pad layer (layer 1), a plasma separation membrane layer (layer 2), a conjugate layer (layer 3), an incubation layer (layer 4), a test read-out layer (layer 5), and blotting layer (layer 6). Vertical flow, rather than lateral flow, was used to wick fluid in the disclosed six-layer device. One advantage of this design is that the effects of gravity in the vertical flow arrangement allow for a shorter diagnosis time of the targeted analyte. In addition, it eliminates potential hook effect problems that could compromise the detection efficiency of the diagnostic test.

### Example 6

### Preparation of Devices for Immunoassays (Treatment of Hydrophilic Regions of Conjugate and Test Read-Out Layers)

Preliminary studies for the detection of the FK-506 analyte were conducted to determine the precise conditions for amplifying the signal obtained from positive samples. This helps to eliminate false positive results arising from non-specific binding when the device is challenged with differentiating negative samples from positive samples. The sample pad layer, incubation layer and blotting layer were not treated. First, the conjugate layer was treated with the solution that contained the surfactant and blocking agent, and was allowed to air dry at room temperature. This was followed by treating the layer with colloidal gold anti-FK-506 detection antibody solution. The test read-out layer (nitrocellulose membrane), was treated with the surfactant and air dried. BSA-FK-506 solution was then added to the sample test zone located in this readout layer. In addition and as shown in FIG. 3B, the Rabbit Anti-Mouse IgM solution was added to the positive control test zone (also located in this layer) followed by the blocking solution. The device was then assembled in preparation for testing.

### Example 7

### Immunoassay Implementation

Whole human blood samples spiked with tacrolimus concentrations at about 100 ng/mL, about 25 ng/mL, about 21 ng/mL, about 10 ng/mL, about 4 ng/mL, about 1 ng/mL, and about 0 ng/mL were tested in the device. The immunoassay was initiated by adding about 20 μL of a sample to the device sample pad layer. The sample was permitted to be completely adsorbed into the top layer of the device, which was followed by immediately adding about 60 μL of wash buffer. The wash buffer volume was three times higher than the volume of the tested sample to eliminate false positives that could have resulted from the blood components present in the unmodified spiked human blood samples. The results were determined by peeling the devices' layers apart to expose the test read-out layer. This allows for color interpretation by the naked eye. The red color formation for tacrolimus detected at these concentrations was quantified using the NIH ImageJ software for images taken by an Android phone. As shown in FIG. 1C, the grey intensity was quantified, and statistical analysis of the results was performed. The detection time was less than about 10 minutes for all the assays performed. Triplicate experiments were performed for each concentration.

### Example 8

### Shelf Life of Device

As previously disclosed in Examples 6 and 7, devices were prepared and assembled to test the shelf life of the paper-based POC device when stored for 15 days at about 50° C. The devices were stored at about 50° C., and testing was performed at days 0 and 15. Whole human blood spiked with about 10 ng/mL tacrolimus was used to test the shelf life of the devices. The results were quantified, and statistical analysis was performed to determine if there was a significant difference between the results on days 0 and 15. Triplicate experiments were performed for each condition.

### Example 9

### Effects of Interferents on Device Assay Performance

To characterize the effects of potential interferents on the device assay performance, devices were prepared and assembled as disclosed in Examples 6 and 7. The potential sources of interference that were tested were sirolimus (rapamycin), mycophenolate mofetil, bilirubin, cholesterol, uric acid, albumin, and gamma globulin. First, tacrolimus was tested in the presence of commonly co-administered drugs to determine whether these drugs would have any interference in the quantification of tacrolimus when assayed by the diagnostic device disclosed herein. We spiked whole human blood with about 10 ng/mL of tacrolimus, about 300 ng/mL sirolimus (rapamycin), and about 100,000 ng/mL mycophenolate mofetil. This mixture was then tested in the device disclosed herein. In addition, we tested tacrolimus in the presence of a mixture of interference endogenous substances. In this test, whole human blood was spiked with about 10 ng/mL of tacrolimus, about 0.6 mg/mL bilirubin, about 5 mg/mL cholesterol, about 0.2 mg/mL uric acid, about 120 mg/mL albumin, and about 120 mg/mL gamma globulin. The results were quantified, and statistical analysis was performed to determine if there was a significant difference between the results of devices tested with tacrolimus alone and devices tested with tacrolimus co-administered with other interferent substances. Triplicate experiments were performed for each condition.

### Example 10

### Statistical Analysis

The statistical analyses were performed by using GraphPad Prism (La Jolla, Calif., U.S.A.). All the statistical data was determined by Paired and Unpaired t-test. In this work, the data was represented as an average±standard deviation (*p<0.05, **p<0.01, ***p<0.001, and ****p<0.0001).

The section headings used herein are for organizational purposes only and are not to be construed as limiting. While the applicant's teachings are described in conjunction with various embodiments, it is not intended that the applicant's teachings be limited to such embodiments. On the contrary, the applicant's teachings encompass various alternatives, modifications, and equivalents, as will be appreciated by those of skill in the art. Accordingly, it will be understood that the invention is not to be limited to the embodiments disclosed herein, and is to be understood using the following claims, which are to be interpreted as broadly as allowed under the law.

What is claimed is:

1. A device for performing a competitive immunoassay for detecting tacrolimus, comprising:
a plurality of layers each comprising one or more hydrophilic regions, one or more hydrophilic channels, or a combination thereof embedded in the layers,
wherein the one or more hydrophilic channels are fluidically connected to the one or more hydrophilic regions;
wherein the plurality of layers comprises a sample pad layer, a plasma separation membrane layer, a conjugate layer, an incubation layer, a test read-out layer, and a blotting layer,
wherein the conjugate layer comprises at least two hydrophilic regions each comprising colloidal gold conjugated with anti-FK-506 antibodies, and
wherein the test read-out layer comprises at least a first hydrophilic region and a second hydrophilic region, wherein the first hydrophilic region comprises a first reagent, and wherein the second hydrophilic region optionally comprises a second reagent selected from the group consisting of antigens and antibodies.

2. The device of claim 1, wherein the fluid sample is a serological sample.

3. The device of claim 2, wherein the serological sample is a blood sample.

4. The device of claim 1, wherein the one or more of the layers are cellulose-based layers.

5. The device of claim 1, wherein the first reagent from the first hydrophilic region of the read-out layer is BSA-FK 506 conjugate.

6. The device of claim 1, wherein the second reagent from the second hydrophilic region of the read-out layer is an antibody, and wherein the antibody is an anti-IgM antibody.

7. The device of claim 1, wherein the two hydrophilic regions of the conjugate layer further comprise a first buffer, wherein the first hydrophilic region of the read-out layer comprises a second buffer, and wherein the second hydrophilic region of the read-out layer comprises a third buffer.

8. The device of claim 7, wherein the first, second, and third buffers comprise DPBS buffer.
